(19) United States
(12) Patent Application Publication
(10) Pub. No.: US 2007/0154560 A1
(43) Pub. Date: Jul. 5, 2007

(54) Process for producing microsphere and apparatus for producing the same

(75) Inventor: Suong-Hyu Hyon, Kyoto (JP)

Correspondence Address: JORDAN AND HAMBURG LLP, 122 EAST 42ND STREET, SUITE 4000, NEW YORK, NY, 10168, US

(73) Assignee: MG Pharmacy Inc., Uji-shi (JP)

(21) Appl. No.: 10/584,719

(22) PCT Filed: Dec. 24, 2003
(86) PCT No.: PCT/JP03/16590

§ 371 Date: Jun. 23, 2006

(51) Int. Cl. A61K 9/14; B29B 9/00

(52) U.S. Cl. 424/489; 264/5

(57) ABSTRACT

A process and apparatus for producing microspheres containing an active component as to be releasable, the process comprising: preparing polymer solution or dispersion having at least active agent, solvent or dispersant, and polymer; drop-wise spitting the polymer solution or dispersion into a flowing fluid, at a predetermined temperature, as to form microsphere precursors; and allowing transfer of the solvent or dispersant within the microsphere precursors to the fluid on way of transporting the microsphere precursors held in the flowing fluid. The process is based on a technique that differs completely from conventional processes of producing microcapsules and nanospheres.

### FIELD OF THE INVENTION

The invention relates to a process and an apparatus for producing microspheres, on basis of a technique that differs completely from conventional processes of producing microcapsules and nanospheres. In particular, the invention relates to a process and an apparatus for producing microspheres that contain active substances as releasable to outside of the polymer sphere. The active substances are preferably pharmaceutical substances; and the invention-wise process is suitable for producing microspheres that are intended to be used in drug delivery systems (DDS) or the microspheres for the DDS.

### BACKGROUND OF THE INVENTION

Many reports and proposals have been made on dosage form and manufacturing process of the various microcapsules, microspheres or ribosome. Most of them are by a concept of the drug delivery systems (DDS) aimed for release control, target selectivity or directivity, easy administration, or improvement of beneficial effect and suppression of side effect or the like.

Among conventional processes for obtaining micro capsules or spheres, predominantly employed are: interfacial precipitation or in-liquid drying by forming “water-in-oil-in-water” (W/O/W) emulsion, as in JP-1967-13703B (Japan's examined patent publication No. S42-13703) for example; phase separation by use of coacervation agents, as in JP-1982-118512A (Japan's patent application publication No. S57-118512) for example; and interfacial polymerization technique, and the like. The conventional processes have problems in that; control of size and its distribution of the capsules or spheres is difficult and poor in reproducibility; pharmaceutical substances would diffuse out to outer aqueous phase on the in-liquid drying; or the capsules would agglomerate together by way of fusion. Moreover, excessive initial-phase release of the pharmaceutical substances, or so-called initial-phase bursting release would take place; and in view of this, it is desired to design drug products as to have ideal gradual release performance by way of “zero-order release” of pharmaceutical substances.

Hence, it is necessary first of all to investigate and review structures, properties and performances of the micro capsules or spheres, in a viewpoint of whether they are really adequate for achieving the functions and performances of the DDS as intended. Even when excellent ones of the micro capsules or spheres are obtained, if its manufacturing requires complicated processes or induces low production yield or problems in safety, production cost will be accordingly increased as to make an obstacle for practical application.

As in the above, a desirable effect of the DDS would not be obtainable unless optimization is made on each of the aspects such as; uniformity of shape and size of the micro capsules or spheres, varieties and content (percentage) of the pharmaceutical substances, manner of storage of them at inside of the capsules or spheres, and manufacturing method.

In view of the above, present inventors have come to the instant invention based on a technique that completely differs from conventional processes for obtaining the microcapsules or nanospheres, as a result of intensive investigation. Invention-wise process for producing the microsphere will solve the above problems and be able to cope with various microcapsules and nanospheres and various active substances, while having versatility in application on wide range of ways of usage.

It is aimed to provide a process and apparatus for producing the microsphere in high quality with low production cost, by use of simple equipment based on drastically unconventional technique as to solve the above problems, especially for producing the microspheres to be used in the DDS.

### SUMMARY OF THE INVENTION

Invention-wise process for producing microspheres that contain active component within polymer spheres as releasable, comprises: preparing polymer solution or dispersion having at least active agent, solvent or dispersant, and polymer; drop-wise spitting the polymer solution or dispersion into a flowing fluid, at a predetermined temperature, as to form microsphere precursors; and allowing transfer of the solvent or dispersant within the microsphere precursors to the fluid on way of transporting the microsphere precursors held in the flowing fluid. The fluid is lipophilic one if the polymer is hydrophilic; and the fluid is hydrophilic one if the polymer is lipophilic. Preferably, the fluid is on before hand cooled under a predetermined temperature. The drop-wise spitting of the polymer solution or dispersion may be made continuously with low flow rate as to form the liquid drops, or intermittently by each small amount at a predetermined interval. The spitting of the polymer solution or dispersion is made in a manner to form a predetermined angle in a range of 45-90 degree between a flowing direction of the flowing fluid and a direction of the spitting or emitting. The spitting is made through a nozzle. Average diameter of the microspheres is in a range of 0.0001-5000 micro meter. The active component is preferably a pharmaceutical substance having physiological function. The polymer is one selected from a group consisting of: polyvinyl alcohol, polymethyl methacrylate, polyester, polycarbonate, polyurethane, polyurea, polyamide, poly alkylene oxalate, homopolymers of hydroxycarboxylic acids, copolymers of hydroxycarboxylic acids, polyamino acids, cellulose derivatives, dextran derivatives, gelatin, shellac, waxes, chitin, and chitosan. Average molecular weight of the polymer is in a range of about 1000-1000,000. The polymer is preferably in vivo degradable. The solvent or dispersant is one selected from a group consisting of: water, alcohols, esters, halogenated hydrocarbons, ethers, aromatic hydrocarbons, hydrocarbons and ketones. The polymer solution or dispersion has a viscosity in a range of 50-10,000 cP at 25° C. The predetermined temperature is in a range of 4-40° C. The fluid is a liquid that is at least one selected from water, alcohols, acetone, acetonitrile and liquid paraffins, and contains a surfactant at 0.1-10 weight-per-volume (W/V) %. Flow rate of the flowing fluid is a constant rate in a range of 0.1-500 mL/minute.

Invention-wise apparatus for producing microspheres that contain active agent within polymer spheres as releasable, comprises; a main body in which a fluid flows or moves; a fluid supplier that sends out liquid as the fluid so that the liquid moves or flows at a predetermined flow rate in the main body; and a polymer solution spitter that drop-wise spits, into the fluid, the polymer solution or dispersion having at least active agent, solvent or dispersant, and polymer, at a predetermined temperature, as to form microsphere precursors; wherein the solvent or dispersant within the microsphere precursors is transferred to the fluid on way of transportation of the microsphere precursors within the main body. The fluid supplier has a feed tube through which the fluid is sent out into the main body. A plurality of such feed tubes is arranged in a predetermined interval. The polymer solution spitter has a nozzle so that direction of spitting the polymer solution or dispersant into the fluid makes a predetermined angle with a direction of flowing of the fluid. A plurality of such nozzles is arranged in a predetermined interval. The invention-wise apparatus further comprises a temperature keeper by which each of the main body, the fluid supplier and the polymer solution spitter is kept at temperature in a range of 4-40° C. The invention-wise apparatus further comprises; a reservoir for the polymer microspheres at beneath of the main body; and a stirrer for stirring the liquid within the reservoir, which contains the polymer microspheres. The drop-wise spitting of the polymer solution or dispersion may be made continuously with low flow rate as to form the liquid drops, or intermittently by each small amount at a predetermined interval; the fluid is lipophilic one if the polymer is hydrophilic; and the fluid is hydrophilic one if the polymer is lipophilic. The spitting of the polymer solution or dispersion is made in a manner to form a predetermined angle in a range of 45-90 degree between a flowing direction of the flowing fluid and a direction of the spitting or emitting. Average diameter of the microspheres is in a range of 0.0001-5000 micro meter.

### DETAILED DESCRIPTION OF THE INVENTION

The invention is directed to a process and an apparatus for producing microspheres, on basis of a technique that differs completely from conventional processes of producing microcapsules and nanospheres. In following, microspheres, an apparatus for producing the microspheres and process thereof are explained in this order.

<Microspheres>

The microspheres obtainable by the invention-wise process are those having an active agent as to be releasable. Term of the “microsphere” will be referred to micro-size spherical particles of polymer, which encompasses all of those that are called as microcapsules, microspheres, micro particles, nanoparticles, nanospheres, nanocapsules or the like. Aphrase of “containing an active agent as releasable” will indicate that; the active agent is released to outside of the microspheres when reaching a predetermined condition or predetermined lapsed time after administration, application, implementation or intake of the microspheres and until then, the active agent is kept as protected from outside environment.

The microspheres, in which releasing to the outside is controllable, may be preferably used to those intended for the DDS or the like. Performances of the microspheres for the DDS, which are selected from release control, target selectivity or directivity, easiness on intake or administration, improvement of beneficial effect and suppression of side effect, are in principal based on kind, structure, property and the like of the polymer. Average diameter of the microspheres is in a range of 0.0001-5000 μm in general, preferably of 0.01-1000 μm, and more preferably of 0.1-500 μm. The microspheres having narrow distribution of diameters and having substantially perfect spherical shape may be obtained by invention-wise process and apparatus. Optimum range of the diameters is found for each usage in accordance with extent of gradual release and with dosage forms. When applied as injection drug in a form of dispersant for example, the average diameter in a range of about 0.5-400 μm is required for achieving dispersion and syringe-needle passing performances. The above-mentioned range of the average diameters satisfies such requirement. Similarly, the microspheres may be used in other dosage forms for transmucous administration, oral administration, suppository and implanting, without causing problems.

<Apparatus for the Producing>

Invention-wise apparatus for producing microspheres that contain active component within polymer spheres as releasable, comprises; a main body in which a fluid flows or moves; a fluid supplier that sends out liquid as the fluid so that the liquid moves or flows at a predetermined flow rate in the main body; and a polymer solution spitter that drop-wise spits, into the fluid, the polymer solution or dispersion having at least active agent, solvent or dispersant, and polymer, at a predetermined temperature, as to form microsphere precursors; wherein the solvent or dispersant within the microsphere precursors is transferred to the fluid on way of transportation of the microsphere precursors within the main body.

FIG. 1 illustrates one embodiment of the invention-wise apparatus, which is no ways limited to such embodiment. Main body of the apparatus comprises a tube or a main-body tube and a keep-warm device that keeps temperatures of the main-body tube and fluid in it as constant. Shape of the main-body tube is preferably circular cylinder although the other shapes may also be employed. Orientation of the main-body tube determines flowing direction of the fluid and is preferably vertical or in a direction achieving downward flow of the fluid, in general situations. Such main-body tube in vertical orientation may be called as a column; and the column may be formed of any material as long as stable to the fluid. The column is preferably formed of glass, polycarbonate resin, acrylic resin, tetrafluoro ethylene resin or other perfluoro alkyl resin, melamine resin, phenolic resin, epoxy resin, polystyrene resin or the like, in general situations. Diameter of the column may be selected in view of number of to-be-mentioned nozzles and also may be set other ways. The diameter of the column is in a range of about 1-50 cm in general and is preferably in a range of 3-5 cm. Length of the column is in a range of 50-300 cm in general and possibly longer or shorter if sufficiently long for serving as the main-body tube, and is preferably in a range of 50-100 cm. The column may be formed as having an outer-tube jacket for keeping temperature of the fluid.

If necessary, at beneath of the main body of the apparatus, a reservoir for the microspheres is directly connected to bottom of the column or the tube; and a stirrer such as a magnetic stirrer may also be provided for stirring the liquid suspended with the microspheres, in the reservoir. The above-mentioned keep-warm device may also keep temperature of each of; the fluid supplier and the polymer solution spitter at a constant temperature, not only temperature of the main body of the apparatus.

The fluid supplier is preferably has a liquid feed tube through which a liquid is sent out to the main body of the apparatus. Typically, the fluid supplier is comprised of a reservoir for the liquid and a liquid-feeding mechanism. The liquid feed tube connects the fluid supplier and the main body of the apparatus. Through the liquid feed tube, the liquid is sent out to the tube of the main body by a liquid-feeding device such as a pump. When for producing a large amount of microspheres at same conditions and within a short period, a plurality of the liquid feed tubes are provided and arranged at a predetermined interval.

The polymer solution spitter typically has a reservoir for storing the polymer solution or dispersion, which is sent to the main body through a feeding tube or the like, driven by a liquid-feeding device such as a pump. On end of the feeding tube, a nozzle is provided. Shape and inner diameter of the nozzle are designed so as to drop-wise spit the polymer solution or dispersion in a favorable manner. Diameter of the nozzle is typically very small and in a range from a several micro meters to several millimeters. Spitting the polymer solution or dispersion into the fluid is made so that a direction of the spitting makes a predetermined angle in regard to a direction along which the fluid flow in the main body. Preferably, a plurality of the nozzles is arranged in a predetermined interval; and thereby, large amount of microspheres are simultaneously produced under identical condition within a short period. By a feeding device such as a pump, the spitting of the polymer solution or dispersion may be made continuously with low flow rate as to form the liquid drops, or intermittently by each small amount at a predetermined interval. The spitting of the polymer solution or dispersion is preferably made in a manner to form a predetermined angle in a range of 45-90 degree between a flowing direction of the flowing fluid and a direction of the spitting of the polymer solution or dispersion.

In following, explanation is made on materials for producing the microspheres by use of the invention-wise apparatus.

<•Polymer>

Polymer as a base material for the microspheres may be water soluble polymer, or one hardly soluble to water. The term “hardly soluble” means that solubility of the polymer to the water is more than zero and no more than 1 weight % (W/W). Polymers compatible to living body are preferred; and natural polymers as well as synthetic polymers may be adopted. Polymers adoptable for producing the microspheres include; polymers of vinyl alcohol, olefins, styrene, vinyl chloride, vinyl acetate, vinylidene chloride, vinyl ethers, vinyl esters, acrylic ester, methacrylic ester, acrylonitrile, methacrylonitrile or the like; polycarbonate, polyurethane, polyurea amide, polyamide, polyacryl amide, poly-α-cyano acrylate esters, copolymers of maleic anhydride, ethylene-vinyl acetate copolymers; polyalkylene oxalates such as poly trimethylene oxalate and poly tetramethylene oxalate; homopolymers and copolymers of hydroxyl carboxylic acids; poly amino acids such as poly-L-alanine, poly-γ-benzyl-L-glutamic acid, and poly-γ-methyl-L-glutamic acid; cellulose derivatives such as acetyl cellulose and nitro cellulose; dextran derivatives, agar, albumin, collagen, casein, gelatin, pectin, shellac, waxes, alginate; natural gums such as gum arabic and karaya gum; chitin and chitosan. Especially preferable among these are in vivo degradable polymers that are physiologically non-active and decomposed to disappear in a living body rather rapidly. Biodegradable polyesters are exemplified by; the homopolymers and copolymers of hydroxyl carboxylic acids or their mixture; and polycyanoacrylate. Detailed examples of such polyhydroxyl carboxylic acids are polylactic acid, poly glycol acid, lactate-glycolate copolymer, polycaprolactone, polyhydroxy butyrate, polyhydroxy isobutyrate, polyhydroxy valerate, poly-γ-hydroxy valerate and the like. Especially preferable polymers are lactate-glycolate copolymer, polylactic acid, lactate-caprolactone copolymer, chitin, chitosan and gelatin. The adoptable polymers may be either of; homopolymer, copolymer of two or more kind of monomers, mixture thereof, and their salts. Biocompatible or in vivo degradable polymers to be used in the invention-wise process may be easily synthesized by a conventional process.

When a copolymer of lactic acid and glycolic acid is adopted as the polymer for the microspheres, weight ratio of the lactic and glycolic acids is preferably in a range of 100/0-50/50 (W/W). Weight average molecular weight of the polymer is preferably in a range of about 5,000-30,000 and more preferably in a range of about 5000-20,000. As for glycolate/2-hydroxybutyrate copolymer, weight ratio of glycolic and 2-hydroxybutyric acids is preferably in a range of 40/60-70/30; and weight average molecular weight of this polymer is preferably in a range of about 5,000-25,000 and more preferably in a range of about 5000-20,000. When a copolymer of butyric and glycolic acids is adopted, weight ratio of the butyric and glycolic acids is preferably in a range of 100/0-25/75 (W/W). When a mixture of polylactic acid (“A”) and the glycolate/2-hydroxybutyrate copolymer (“B”) is adopted, monomer mixing ratio “A/B” is preferably in a range of about 10/90-90/10, and more preferably in a range of about 25/75-75/20. Weight average molecular weight of the polylactic acid is preferably in a range of about 5,000-30,000 and more preferably in a range of about 6,000-20,000. Manner of copolymerization may be either of random, block or grafting. When there are D-enantiomer, L-enantiomer and D,L-enantiomer for a certain hydroxycarboxylic acid, any of the enantiomer may be adopted while D,L-enantiomer is preferred.

Weight average molecular weight of the polymer for the microspheres is preferably set in a range of about 1,000-1,000,000, and more preferably in a range of about 5000-500,000.

<•Solvent or Dispersant>

Any solvent or dispersant may be employed for the polymer solution or dispersant as long as being a good solvent or dispersant for the polymer. For example, the solvent or dispersant is one selected from a group consisting of: water, alcohols, esters, halogenated hydrocarbons, ethers, aromatic hydrocarbons, hydrocarbons and ketones. Specifically mentionable are; methanol, ethanol, propanol, ethyl acetate, butyl acetate, dichloromethane (methylene chloride), chloroform, carbon tetrachloride, chloroethane, dichloroethane, trichloroethane, dichlorohexane, ethyl ether, isopropyl ether, tetrahydrofuran, methoxy ethyl ether, 1,4-dioxane, benzene, toluene, xylene, n-pentane, n-hexane, acetone, methyl-ethyl ketone, acetonitrile and so on. When polylactide or lactide-glycolate copolymer is used as the polymer, ethyl acetate or dichloromethane is preferred.

<•Fluid>

The fluid is contained in the fluid supplier and sent into the main body of the apparatus, through a fluid feeding tube and flows in a main-body tube at a predetermined flow rate. Into such stream of the fluid, the polymer solution or dispersant is drop-wise spited so as to form precursors of the microspheres. Hence, the fluid takes a role of carrier or perfusion liquid that transport the precursors. The fluid is selected from the above-mentioned solvent or dispersant in a manner that the fluid is lipophilic one if the polymer is hydrophilic; and the fluid is hydrophilic one if the polymer is lipophilic. By thus setting lipophilic-hydrophilic properties of the fluid and the polymer as opposite to each other; the microspheres may be produced not only from the polymer hardly soluble to water but also from water soluble polymer. Adoptable as the fluid are; water, alcohols, acetone, methanol, ethanol, tetrahydrofuran, ethyl acetate, acetonitrile, acrylonitrile, liquid paraffin and so on. When selecting the fluid from these solvents, the above relationship of lipophilic-hydrophilic attributes of the fluid and the polymer has to be taken into consideration. In view of easiness of handling, the fluid is preferably at least one selected from a group consisting of; water, ethanol and liquid paraffin. In view of safety, water and liquid paraffin are especially preferred. The microspheres of dextrin or gelatin, which are water-soluble, may be produced as follows for example; liquid paraffin is employed as the fluid and its temperature is controlled so that removal of water from microspheric bodies is achieved during their precipitation in the fluid.

The fluid contains 0.1-10% of surfactant in general situations, for forming drops of the polymer solution or dispersant, and preferably contains 1-3% of surfactant. Any surfactant may be used so long as it is in general use. Mentionable surfactants are; sorbitan fatty acid ester, polyoxyethylene sorbitan fatty acid ester, glycerin fatty acid ester, polyoxyethylene hydrogenated castor oil, polyoxyethylene alkylethers, sodium lauryl sulfate, sodium oleate, sodium stearate, polyvinyl alcohol, polyvinyl pyrrolidone, lecithin, carboxymethyl cellulose and soon. The fluid is kept at a constant temperature in a range of 4-40° C., preferably in a range of 10-40° C., and preferably by operation of the keep-warm device that keeps the fluid supplier and the main body of the apparatus at a constant temperature. The flow or moving rate of the fluid is a constant rate in a range of 0.1-500 mL/minute in general and preferably in a range of 0.5-50 mL/minute.

<•Active component>

Active component enclosed within the microspheres is typically a pharmaceutical substance, and may contain additional substances such as auxiliary agents and stabilizers as the need arises. The active component may be chemicals in wider range other than the pharmaceuticals, and may be chemicals such as agricultural chemicals and fertilizer. The active component may be chemicals in further wider range other than physiologically active substances and may be organic and inorganic substances in wide variety. When range of the active component is expanded in this way, the invention-wise microspheres may be used in wide range of products such as photography chemicals, carbon copy paper, adhesives and paints.

Any physiologically active substance may be enclosed within the microspheres in accordance of specific usage. Hence, the chemicals to be enclosed may be water soluble one and may be one hardly soluble in water. Plurality of chemicals may be enclosed within the microspheres as to stay there together with each other. For example, two, three or four pharmaceutical substances have been simultaneously used in medication for gastric ulcer, pneumonia, common cold and the like as to achieve synergic or complementary effect. Mentionable pharmaceutical substances are; anti-tumor antibiotics, antipyretic analgesics, anti-inflammatory agents, cough suppressant and expectorant drugs, anti-tumor agents, analgesics, muscle relaxant, antidepressant drugs, antiepileptic drugs, anti-tubercular agents, anti-arrhythmic agents, vasodilators, heart stimulants, anti-allergic agents, anti-hypertensive diuretic agents, diabetic medicine, anticoagulants, hemostatic agents, hormone drugs, physiologically active peptides, vessel-growth depressants, vascular reinforcers, narcotic antagonists, bone resorption depressant rheumatics, contraceptives, choleretics or liver support agents, stomachic digestive aids, antiflatuent or bowel medicine, vitamin supplements, vaccine formulations, constipation-relieving agents, hemorrhoid drugs, varieties of enzyme formulations, antiprotozoas, interferon inducers, insectfuges, exodermic sterilizers, parasitic dermatitis drugs and so on. Specifically mentionable as adoptable pharmaceutical substances are as below, while below listing by no means does not restrict scope of the invention.

Mentionable as antitumor agents are; methotrexate, actinomycin D, mitomycin C, bleomycin hydrochloride, vinblastine sulfate, vincristine sulfate, adriamycin, neocarzinostatin, fuorouracil, cytosinearabinoside, krestin, picibanil, lentinan, bestatin, levamisole, azimexon, glycyrrhizin, cisplastin and so on. Mentionable as antibiotics are; tetracycline hydrochloride, oxytetracycline hydrochloride, doxycylinen hydrochloride, rolitetracycline, amikacin, fradiomycin, sisomicin, gentamicin, bekanamycin sulfate, dibekacin, lividomycin, tobramycin, ampicillin, amoxicillin, ticarcillin, piperacillin, cephaloridine, cephalothin, cefsulodin, cefotiam, cefmenoxime, cefmetazole, cefazolin, cefotaxime, cefoperazone, ceftizoxime, moxolactam, “furufazesin” (in Japanese), aztreonam, thienamycin, etronidazole, clarithromycin and so on. Mentionable as antipyretic analgesic and anti-inflammatory agents are; sodium salicylate, sulpyrine, diclofenac sodium, sodium fulfenamate, indomethacin sodium, morphine hydrochloride, pitidine hydrochloride, oxymorphant, levorphanol tartrate and so on. Mentionable as antilussive expectorants are; ephedorine hydrochloride, methyl ephedorine hydrochloride, noscapine hydrochloride, codeine phosphate, dihydrocodeine phosphate, clofedianol hydrochloride, allocramide hydrochloride, picoperidamine, cloperastine, isoproterenol hydrochloride, protokylol hydrochloride, salbutamol sulfate, terbulaline sulfate and so on.

Mentionable as anti-ulcer agents are histidine hydrochloride and metoclopramide; mentionable as sedative drugs are prochlorperazine, chlorpomazine hydrochloride, trifluoperazine, atropine sulfate and methylscopolamine bromide; mentionable as muscle relaxants are pancuronium bromide, tubocurarine hydrochloride, tubocurarine hydrochloride and pridinol mesylate; mentionable as antidepressant are imipramine, clomipramine, noxiptyline, phenelsine sulfate; and mentionable as antiepleptic agents are chlordiazepoxide hydrochloride, acetazolamide sodium, phenytoin sodium and ethosuximide. Mentionable as diabetic drugs are phenformin hydrochloride, glymidine sodium, buformin hydrochloride and glipizide; mentionable as anticoagulants are heparin sodium and sodium citrate; mentionable as hemostats are thrombin, thromboplastin, acetomehaphtone, menadione sodium hydrogen sulfite, tranexamic acid, ε-aminocaproic acid, adrenochrome monoamino guanidine methanesulfonic acid, and carbazochrome sodium sulfonate.

Mentionable as antituberculous agents are sodiumparamino salicylate, ethambutol and isoniazid. Mentionable as antiarrhythmic agents are propranol hydrochloride, alprenolol hydrochloride, bufetolol hydrochloride, oxyprenolol hydrochloride. Mentionable as vasodilator are diltiazem hydrochloride, oxyfedrine hydrochloride, trazoline hydrochloride, hexobendine and bamethan sulfate. Mentionable as cardiac stimulants are aminophylline, theophyllol, etilefrine hydrochloride and trans-bioxocamphor. Mentionable as antiallergic agents are chlorpheniramine maleate, methoxyphenamine hydrochloride, diphenhydramine hydrochloride, tripelenamine hydrochloride, methdilazine hydrochloride, clemizole hydrochloride, methoxyphenamine hydrochloride, diphenylpyraline hydrochloride. Mentionable as antihypertensive diuretic agents are pentolinium, hexamethonium bromide, mecamylamine hydrochloride, ecarazine hydrochloride and clonidine hydrochloride. Mentionable as hormone formulation are prednisolone sodium phosphate, mprednisolone succinate, dexamethazone sodium sulfate, betamethazone sodium sulfate, hexestrol phosphate and methimazole. Mentionable as anti-angiogenic agents are; fumagillin, fumagillol derivatives, and anti-angiogenic steroids. Mentionable as antinarcotics are nalorphine hydrochloride, naloxone hydrochloride and levallorphan tartrate. Mentionable as bone resorption depressant are aminomethylene-bis-phosphonate and sulpher-containing alkyl aminomethylene-bis-phosphonate. These agents may be used as it is or in a form of salt or derivative.

The physiologically active peptide may be either of oligopeptide and polypeptide so long as having the physiological activity, and preferably has molecular weight in a range of about 200-80,000. Specifically mentionable as such peptides are luteinzing hormone releasing hormone and its derivatives, insulin, somatostatin and its derivatives, growth hormone, prolactin, adrenocorticotropic hormone, melanocyte-stimulating hormone, parathyroid hormone, vasopressin, oxytocin, calcitonin, glucagon, gastrin, secretin, cholecystokinin, pancreozymin, angiotensin, enkephalin, protein-synthesis-stimulating hormone, human chorionic gonandotropin, human placentral lactogen, luteinzing hormone, follicle-stimulating hormone, varieties of interferon, interleukin, endorphin, kyotorphin, tuftsin, thymopoietin, thymosin, thymus thymuline, thymic factor, tumor necrosis factor, colony-inducing factor, nerve growth factor, substance P, kallikrein, motillin, dynorphin, bombesin, cerulein, bradykinin, asparaginase, urokinase, lysozyme chloride, polymyxin B, colistin, gramicidin, bacitracin, erythropoietin, platelet-derived growth factor, growth hormone-releasing factor, and epidermal growth factor.

Apart from the pharmaceutical substances, the active component may be; agricultural chemicals such as antimicrobials, herbicides and pesticides; auxin, plant hormone, insect hormone or piscicide.

Particles of the pharmaceutical or other active chemicals, which are to be added into the polymer solution or dispersant, may have any diameter so long as readily enclosed within the microspheres and are preferably prepared as fine particles by; hammer milling, screen milling, ball milling, vibration milling, jet milling, colloidal milling or pounding in a mortar. Diameter of the particles is no more than 1/10 (10%) of diameter of the microspheres, and is preferably no more than 1/100 (1%) of diameter of the microspheres. When the fore-mentioned diameters of the microspheres are taken into consideration, the diameters of the particles of the chemicals are preferably in a range of 0.00001 μm to several dozen micrometers. When the diameter is no more than 10 μm, the microspheres that are uniform and especially small in diameter are obtained. Concentration or content of the active component in the polymer solution or dispersion is in a range of about 0.001-90 weight (W/W) %, preferably in a range of about 0.01-80 weight %, and more preferably in a range of about 0.01-70 weight %.

<Process for Producing>

Invention-wise process for producing microspheres that contain active component within polymer spheres as releasable, comprises: preparing polymer solution or dispersion having at least active agent, solvent or dispersant, and polymer; drop-wise spitting the polymer solution or dispersion into a flowing fluid, at a predetermined temperature, as to form microsphere precursors; and allowing transfer of the solvent or dispersant within the microsphere precursors to the fluid on way of transporting the microsphere precursors held in the flowing fluid. By adopting the fluid having lipophilic-hydrophilic-wise attribute reverse to that of the polymer; the microspheres of water soluble polymers as well as the microspheres of polymer that are hardly soluble to water are obtainable.

<•Polymer Solution or Dispersion>

The polymer solution or dispersion contains at least the active component, the polymer, and solution or dispersant; and may contain other substances such as auxiliary agents and stabilizer. Solution of the polymer is preferred whereas uniform dispersion of the polymer may also be used in the producing of the microspheres. For obtaining the solution or uniform dispersion, adoptable are; agitators such as magnetic stirrers, propeller agitators and turbine agitators; intermittent shaking, colloidal mills, dispersers, and ultrasonic irradiation. The polymer solution or dispersion is stored in the polymer solution spitter and is maintained at a constant temperature, preferably by the fore-mentioned keep-warmer, preferably in a range of 4-40° C., more preferably in a range of 10-40° C. Flow rate of the polymer solution or dispersion within the polymer solution spitter into the nozzle is a constant rate in a range of 0.1-500 mL/miniute in general and preferably in a range of 0.5-50 mL/miniute. The polymer solution or dispersion is spitted or emitted from the nozzle aperture into the fluid flowing in the main-body tube of the apparatus so as to form a predetermined angle between a direction of the spitting and a direction of flowing of the fluid, in arange of 45-90 degrees. The predetermined angle is determined in a way to optimize formation of drops under given conditions. The spitting of the polymer solution or dispersion may be made continuously with low flow rate as to form the liquid drops, or intermittently by each small amount at a predetermined interval. The spitting has to be made in a manner to throw the drops of the polymer solution or dispersion into the fluid, so that the drops as the precursors are transported by the flowing fluid and to form the microspheres having uniform diameters.

When a plurality of nozzles arranged at a predetermined interval is provided for the polymer solution spitter, a large amount of the microspheres are produced under identical conditions within a short period. Concentration or content of the polymer in the polymer solution or dispersion is preferably in a range of 1-50 weight-per-volume (W/V) % and more preferably in a range of10-40W/V %. When the concentration is less than 1 W/V %, ratio of the active chemicals that are included or distributed within the microspheres becomes too small. When the concentration is more than 50 W/V %, there arises problems such as difficulty in forming the microspheres. In place of the above, ratio of the polymer to the fluid is preferably in a range of 99.9/0.1 to 50/50 and more preferably in a range of 99/1 to 70/30. When the polymer solution or dispersion is diluted to be out of this range, viscosity formation within the precursors transported by the flowing fluid becomes insufficient so that leaking out of the active chemicals to be included in the microspheres becomes too large, and thus, the ratio of the active chemicals that are included or distributed within the microspheres becomes too small. When the polymer solution or dispersion is denser to be out of the above range, drops of the polymer solution or dispersion become too large so that viscosity formation of the precursor might become too small. In order to form preferable micro-size liquid drops in the fluid, viscosity of the polymer solution or dispersant is preferably in a range of 50-10,000 cP, more preferably in a range of 200-2000 cP. When the viscosity is less than 50 cP, the polymer solution or dispersion might not become micro-size drops during transportation on the stream of the fluid. The viscosity more than 10,000 cP might cause too large size of the drops of the polymer solution or dispersion.

<•Forming of the Precursors and the Microspheres>

The polymer solution or dispersion is spitted into the flowing fluid continuously with low flow rate as to form the liquid drops, or intermittently by each small amount at a predetermined interval. During transportation of the liquid drops in the fluid, the liquid drops by its surface tension form the precursors having uniform diameters. During such transportation, the solvent or dispersant migrates to the fluid to a greater or less extent. Migration or transfer amount of the solvent or dispersion is submissive to various factors such as temperature, the polymer solution or dispersion, the fluid, flowing rate of the fluid or the like. The fluid and the polymer solution or dispersion are always kept in a temperature range of 4-40° C., preferably at a constant temperature in a range of 10-40° C. During transportation of the precursors of the microspheres and migration of the solvent or dispersant migrates into the fluid, content of the solvent or dispersant within the microspheres decreases, especially in its polymer part, thereby causing hardening of the polymer parts. The polymer part forms outer shell by a kind of drying-wise solidification as to make a state of enveloping the active chemicals or similar state, thereby completing formation of the microspheres. The microspheres obtainable by the invention-wise method are substantially complete spheres having a narrow distribution of diameters as uniform particles.

<•Handling or treating of the product>

The microspheres formed during transportation in the flowing fluid are collected in the reservoir for the polymer microspheres at beneath of the main body and are agitated by an agitating device in a last process steps for “encapsulation” or forming the microspheres enclosing the active chemicals. On course of such process, solvent or dispersant is removed from microspheric particles and the microspheres having strength and a narrow distribution of the diameters. Time period required to achieve such stable state is in a range of 0.5-2 hours in general and preferably in a range of 1-1.5 hours. By such way of achieving the stable state, the polymer agglomerates in a compacted manner, so that no porous structure is formed after removal of the solvent and surface structure curbing the “initial-phase bursting” release is formed. Thus produced microspheres are collected by centrifugation or filtration and then washed with distilled water or other adequate solvents. If necessary, solvent or water or the like in the microspheres is completely removed by vacuum drying, freeze drying or the like.

The microspheres obtained by the invention-wise process are able to contain the active chemicals irrespective to whether the active chemical is soluble or hardly soluble in water. The polymer is selected in accordance of usage of the microspheres; and controlling of diameters of the microspheres is easily made. Thus, the microspheres may be used in wide variety of pharmaceutical products, and may be used for example in hypodermic, endermic, intramuscler and intraperitoneal or oral administration of pharmaceutical or its applying in the affected part, or in the vein or artery. The microspheres are administered or applied after dispersed in a dispersant in general situations. When for gradual release of agricultural chemicals, the microspheres are sprayed on soil or foliage of the plant. The invention-wise process, which enables controlling of diameters of the microspheres and thickness of their shells or membranes in a wide range, is promising in usage of functional microcapsules or microspheres containing protein, enzyme, antibody, genes (DNA or RNA) or the like.

### BRIEF DESCRIPTION OF THE DRAWINGS

FIG. 1 shows an embodiment of the invention-wise apparatus for producing the microspheres, in which a fluid supplier 1, a polymer solution spitter 2, a spray nozzle 3, a drain 4, a column 5 filled with a fluid, a reservoir 6 for the microspheres and a magnetic stirrer 7 are appeared;

FIG. 2 shows micrographs of the microspheres obtained by process-wise Example, in which left-side photo is taken by an optical microscope and right-side photo is taken by a scanning electron microscope;

FIG. 3 shows micrographs of the microspheres obtained by process-wise Comparative Example, in which left-side photo is taken by an optical microscope and right-side photo is taken by a scanning electron microscope;

FIG. 4 is a graph showing release of taxol from the microspheres obtained by the process-wise Example, in which ordinate indicates percentage (%) of released one and abscissa indicates period or days; and

FIG. 5 is a graph showing release of taxol from the microspheres obtained by the process-wise comparative example 1, in which ordinate and abscissa indicates those as in FIG. 4, and in which solid square mark indicates an occasion where acetic acid is used as solvent for the polylactic acid and solid triangle mark indicates an occasion where acetonitrile is used as such solvent.

### EMBODIMENTS

In following, embodiments of the invention are shown by way of explaining the invention in detail, while the description below by no means to restrict scope of the invention.

Following materials or reagents were used for the process-wise examples at below. L-lactate/glycolate copolymer (PLGA) having molecular weight of 18,000 and monomer ratio of 75 (L-lactate)/25(glycolate) is obtained from BMG Incorporated in Kyoto, Japan; poly vinyl alcohol (PVA) having a saponification degree of 88% and polymerization degree of 250 was obtained from UNITIKA Ltd., in Osaka, Japan; taxol was purchased from Sigma Co.; and a high-pressure liquid chromatography device of TOSOH Corporation was used.

<Process-Wise Example>

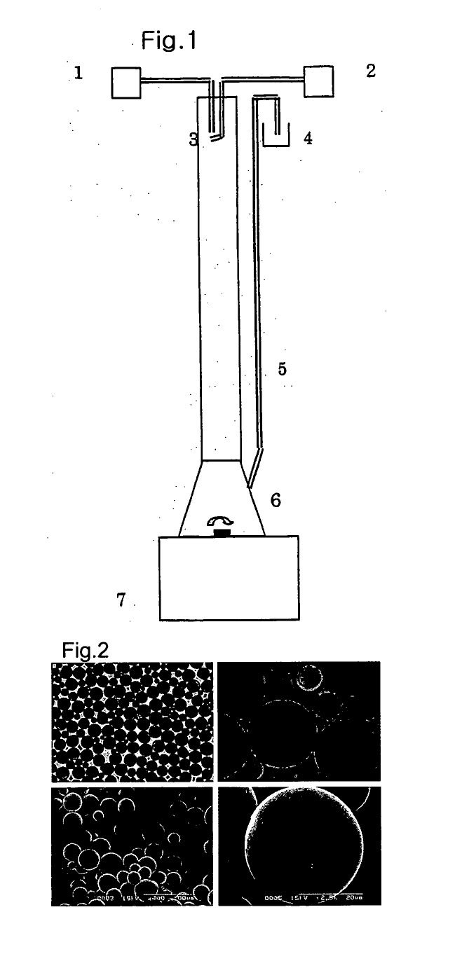

Five milliliter of 30% solution of the PLGA in ethyl acetate solvent is prepared; and taxol is simultaneously dissolved on preparing the PLGA solution, in a ratio of 10weight % of taxol to the PLGA, in order to achieve inclusion of taxol in the polymer. An apparatus for producing the microspheres as shown in FIG. 1 is used; and the PLGA solution dissolved with taxol is spitted or emitted at a flow rate of about 0.2 mL/minute through a syringe needle of 0.5 mm diameter by use of a cassette tube pump. A fluid as carrier, that is 1% PVA solution in distilled water, is supplied by a pump as to make a flow in a direction having an angle of 90 degree with respect to a direction of flowing of the PLGA solution. From the syringe needle as a nozzle, uniform drops of the solution fall down in a glass tube in a length of 1.5 m, which is filled with the 1% PVA solution, and reach a collector bottle that is a reservoir. Inside of the collector bottle or the reservoir is stirred with a magnetic stirrer. Thus collected microspheres are washed with distilled water repeatedly three times and then freeze-dried.

<Process-Wise Comparative Example>

Five mile liter of 8% solution of the PLGA in ethyl acetate solvent is prepared; and taxol is simultaneously dissolved on preparing the PLGA solution, in a ratio of 5 weight % of taxol to the PLGA, in order to achieve inclusion of taxol in the polymer. The PLGA solution dissolved with taxol is added with liquid paraffin containing 10% of “Span 80”; and then stirred at 260 rpm and heated at rate of 0.1° C./minute from 35° C. to 42° C. After continuing the stirring for 48 hours, obtained microspheres are collected, washed with hexane repeatedly three times and freeze-dried. In another way of producing the microspheres, methyl cyanide is used in place of ethyl acetate for dissolving the PLGA; and except for this, same process as above is employed.

<Test-Wise Example 1>

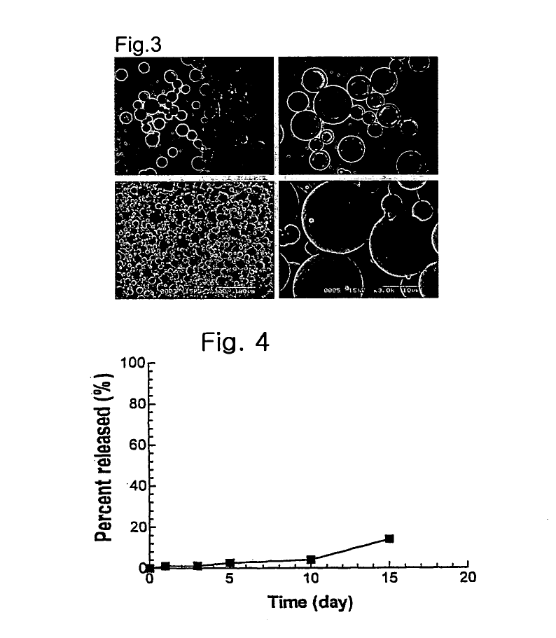

The microspheres obtained by the Process-wise Example and the Process-wise Comparative Example were both suited to be visually examined by an optical microscope. A few drops of the microspheres dispersant is placed on a cover glass and then viewed by an optical microscope of NIKON CORPORATION. The microspheres were coated with gold by sputtering technique and then viewed by scanning electron microscope (“S-4700” of Hitachi, Ltd.) to examine surface and porosity of the microspheres. Micrographs thus obtained are shown in FIGS. 2 and 3.

The microspheres enclosing taxol in both of the Example and the Comparative Example were spherical particles having smooth surfaces. The microspheres obtained by the Process-wise Example had narrower distribution of diameters compared with those obtained by the Comparative Example. Please see FIG. 2. The microspheres of the Comparative Example would easily aggregate with each other as FIG. 3 shows the microspheres attached or partly fused with each other.

<Test-Wise Example 2: Taxol Content in the Microspheres>

Original taxol contents in the microspheres obtained by the Example and Comparative Example were measured by high-performance liquid chromatography (HPLC). The microspheres by amount of 10 mg were weighed with high precision and then dissolved in acetonitrile as to be diluted to be 10 mL. For the HPLC measurement, a reverse phase chromatography system is employed, in which employed are; HPLC columns of “Tosoh ODS” (4.6 ×250 mm); acetonitrile/water solution (60/40) as carrier-phase liquid; detection at 273 nm; and sample injection amount of 20 μL. Results of the HPLC analysis are shown in Table at below.

TABLE
Inclusion of taxol in the microspheres

| | | Comparative |
|---|---|---|
| | Example | Example |
| PLGA Concentration (%) | about 30 | <10* |
| Per-microsphere Taxol Content | 13.2 | 2.7 |
| (weight %) | | |
| Taxol-inclusion ratio (%) | 98 | 54 |

*Microspheres were not obtainable when concentration of the PLGA was no less than 10%, for the Comparative Example.

<Test-Wise Example 3: In vitro Release Test>

Rate of release from the taxol-enclosing microspheres of the Example and Comparative Example was evaluated. The microspheres by amount of5 mg were dispersed in 10 mL of phosphate buffer saline (PBS) having 0.1% of “Tween 80” and pH value of 7.4. Thus obtained dispersions for the Example and Comparative Example were placed in an incubator maintained at 37° C. From the dispersions, sampling by amount of100 μL is made at a constant interval, throughout a period of 30 days. For each sampling, taxol concentration in liquid of the dispersion was measured, under conditions same as in the Test-wise example 2 except that detection was made at wave length of 232 nm.

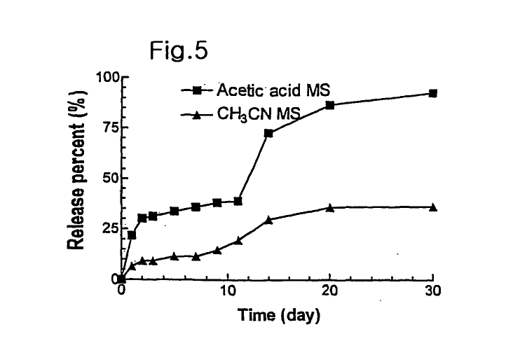

Ratios of released amount of taxol thus obtained are plotted in FIGS. 4 and 5, which are respectively for the microspheres of the Process-wise Example and the Comparative Example. As known from the figures, slow and gradual release of taxol was made by the microspheres of the Process-wise Example; and, by those of the Comparative Example, heavy release of taxol at early stage, or so-called “initial-phase bursting” release, as well as higher rate of over-all release were observed.

### INDUSTRIAL APPLICABILITY

The invention-wise process achieves that active component such as physiological substance is enclosed in a manner as being releasable as well as being uniformly dispersed. Thus, content of the active agent in each of the microspheres is high; percentage of the microspheres enclosing the active component is high; and product yield as a whole is high. The microspheres obtained by the invention-wise process have a narrow distribution of diameters and shapes substantially spherical. The microspheres obtained by the invention-wise process effectively curb the “initial-phase bursting” release of the active component. Thus, its release of physiological substance for a long period in a living body for example is achieved. By the invention-wise process and apparatus, the microspheres enclosing the active component and having a high quality are produced as controlled under identical conditions by simple process steps and low production cost. Moreover, the producing may be made in a wide temperature range and especially in a low temperature range; and use of harmful substance such as cross-linking agent is omitted so that safety is secured. The invention-wise apparatus is applicable to commercial-scale mass production and enables remarkable decrease of production cost because of rather simple construction and processes.

1. A process for producing microspheres that contain active component within polymer spheres as releasable, comprises: preparing polymer solution or dispersion having at least active agent, solvent or dispersant, and polymer; drop-wise spitting the polymer solution or dispersion into a flowing fluid, at a predetermined temperature, as to form microsphere precursors; and allowing transfer of the solvent or dispersant within the microsphere precursors to the fluid on way of transporting the microsphere precursors held in the flowing fluid, wherein the spitting of the polymer solution or dispersion is made in a manner to form a predetermined angle in a range of 45-90 degree between a flowing direction of the flowing fluid and a direction of the spitting.

2. A process for producing microspheres according to claim 1, wherein the fluid is lipophilic one if the polymer is hydrophilic; and the fluid is hydrophilic one if the polymer is lipophilic.

3. (canceled)

4. A process for producing microspheres according to claim 1, wherein the drop-wise spitting of the polymer solution or dispersion is made continuously with low flow rate as to form the liquid drops, or intermittently by each small amount at a predetermined interval.

5-6. (canceled)

7. A process for producing microspheres according to claim 1, wherein average diameter of the microspheres is in a range of 0.1-500 micro meter.

8. (canceled)

9. A process for producing microspheres according to claim 1, wherein the polymer is in vivo degradable and is at least one selected from a group consisting of: poly alkylene oxalate, homopolymers of hydroxycarboxylic acids, copolymers of hydroxycarboxylic acids, polyamino acids, cellulose derivatives, dextran derivatives, gelatin, shellac, waxes, chitin, and chitosan.

10-11. (canceled)

12. A process for producing microspheres according to claim 2, wherein the solvent or dispersant is at least one selected from a group consisting of: water, alcohols, esters, halogenated hydrocarbons, ethers, aromatic hydrocarbons, hydrocarbons and ketones.

13. A process for producing microspheres according to claim 1, wherein the polymer solution or dispersion has a viscosity in a range of 50-10,000 cP at 25° C.

14. A process for producing microspheres according to claim 1, wherein the predetermined temperature is in a range of 4-40° C.

15. A process for producing microspheres according to claim 2, wherein the fluid is a liquid that is at least one selected from water, alcohols, acetone, acetonitrile and liquid paraffins, and contains a surfactant at 0.1-10 weight-per-volume (W/V) %.

16. A process for producing microspheres according to claim 1, wherein flow rate of the flowing fluid is a constant rate in a range of 0.1-500 mL/minute.

17. An apparatus for producing microspheres that contain active agent within polymer spheres as releasable, comprises; a main body in which a fluid flows or moves; a fluid supplier that sends out liquid as the fluid so that the liquid moves or flows at a predetermined flow rate in the main body; a polymer solution spitter that drop-wise spits, into the fluid, the polymer solution or dispersion having at least active agent, solvent or dispersant, and polymer, at a predetermined temperature, as to form microsphere precursors; a reservoir for the polymer microspheres at beneath of the main body; and a stirrer for stirring the liquid within the reservoir, which contains the polymer microspheres; wherein the solvent or dispersant within the microsphere precursors is transferred to the fluid on way of transportation of the microsphere precursors within the main body.

18-21. (canceled)

22. An apparatus for producing microspheres according to claim 17, further comprising a temperature keeper by which each of the main body, the fluid supplier and the polymer solution spitter is kept at temperature in a range of 4-40° C.

23-24. (canceled)

25. An apparatus for producing microspheres according to claim 17, wherein the spitting of the polymer solution or dispersion is made in a manner to form a predetermined angle in a range of 45-90 degree between a flowing direction of the flowing fluid and a direction of the spitting.

26. (canceled)

27. An apparatus for producing microspheres according to claim 17, wherein the main body is a column arranged as vertical or in a direction achieving downward flow of the fluid; and diameter of the column is in a range of 3-5 cm when length of the column is in a range of 50-300 cm.

28. A process for producing microspheres that contain active component within polymer spheres as releasable, comprises: preparing polymer solution or dispersion having at least active agent, solvent or dispersant, and polymer; drop-wise spitting the polymer solution or dispersion into a flowing fluid, at a predetermined temperature, as to form microsphere precursors; and allowing transfer of the solvent or dispersant within the microsphere precursors to the fluid on way of transporting the microsphere precursors held in the flowing fluid, wherein the fluid is a liquid that is at least one selected from water, alcohols, acetone, acetonitrile and liquid paraffins, and contains a surfactant at 0.1-10 weight-per-volume (W/V) %.

29. A process for producing microspheres according to claim 28, wherein the surfactant is at least one selected from a group consisting of sorbitan fatty acid ester, polyoxyethylene sorbitan fatty acid ester, glycerin fatty acid ester, polyoxyethylene hydrogenated castor oil, polyoxyethylene alkylethers, sodium lauryl sulfate, sodium oleate, sodium stearate, polyvinyl alcohol, polyvinyl pyrrolidone, lecithin, and carboxymethyl cellulose.

30. A process for producing microspheres according to claim 29, wherein the polymer solution or dispersion has a viscosity in a range of 200-2000 cP at 25° C.

31. process for producing microspheres according to claim 29, wherein the surfactant is at least one selected from a group consisting of polyvinyl alcohol, polyvinyl pyrrolidone, and carboxymethyl cellulose.

32. process for producing microspheres according to claim 29, wherein the fluid contains a surfactant at 1-3% weight-per-volume (W/V) %.

33. A process for producing microspheres according to claim 28, wherein the spitting of the polymer solution or dispersion is made in a manner to form a predetermined angle in a range of 45-90 degree between a flowing direction of the flowing fluid and a direction of the spitting.

34. An apparatus for producing microspheres that contain active agent within polymer spheres as releasable, comprises; a main body in which a fluid flows or moves; a fluid supplier that sends out liquid as the fluid so that the liquid moves or flows at a predetermined flow rate in the main body; and a polymer solution spitter that drop-wise spits, into the fluid, the polymer solution or dispersion having at least active agent, solvent or dispersant, and polymer, at a predetermined temperature, as to form microsphere precursors; wherein the solvent or dispersant within the microsphere precursors is transferred to the fluid on way of transportation of the microsphere precursors within the main body; and the spitting of the polymer solution or dispersion is made in a manner to form a predetermined angle in a range of 45-90 degree between a flowing direction of the flowing fluid and a direction of the spitting.
